(19) United States
(12) Patent Application Publication
(10) Pub. No.: US 2004/0092753 A1
(43) Pub. Date: May 13, 2004

(54) Method for producing unsaturated cyclic ethers

(76) Inventors: Hagen Weigl, Ladenburg (DE); Klaus Ebel, Lampertheim (DE); Sylvia Huber, Zwingenberg (DE); Andreas Kusche, Kleinfischlingen (DE); Karsten Eller, Ludwigshafen (DE); Michael Hesse, Worms (DE); Arthur Hohn, Kirchheim (DE)

Correspondence Address: Keil & Weinkauf, 1350 Connecticute Avenue NW, Washington, DC, 20036, US

(21) Appl. No.: 10/469,689

(22) PCT Filed: Mar. 6, 2002
(86) PCT No.: PCT/EP02/02425

(30) Foreign Application Priority Data
Mar. 8, 2001 (DE) 101 11 065.0

(51) Int. Cl. C07D 323/02

(52) U.S. Cl. 549/347; 549/356; 549/357; 549/430; 549/429

(57) ABSTRACT

A method for producing unsaturated cyclic ethers of general formula (I) wherein Z represents (CHR⁴)_{q}— or (CHR⁴)_{q}—O—, q 0, 1, 2 or 3 and R¹, R², R³, R⁴ represent hydrogen or C₁-C₄-alkyl, by reacting dioles of general formula (II), wherein Z, R¹, R² and R³ have the above-mentioned meanings, in a liquid phase at temperatures of 150-300° C. in the presence of a support catalyst which is not activated prior to use by reduction and which contains cobalt and is doped with sulphur, containing cobalt, and an earth metal which is deposited by sol impregnation and is selected from the group containing platinum, palladium, rhodium, iridium, ruthenium, osmium, rhenium or mixtures thereof, on an inert support, characterized in that water is added.

### DESCRIPTION

The present invention relates to a process for the production of unsaturated cyclic ethers from diols in liquid phase over supported catalysts doped with sulfur and containing cobalt and noble metal, with the addition of water.

DE-A 2,346,943 describes a process for the production of unsaturated cyclic compounds from diols under a stream of hydrogen, in which the catalysts used are mixtures of a copper chromite catalyst or supported copper catalyst and a tungstic or heteropoly-tungstic acid. The conversions and yields are unsatisfactory.

U.S. Pat. No. 2,993,910 describes a process for the production of dihydrofurans from 1,4-butanediols over cobalt catalysts, which have to be reduced at from 300° to 450° C. with hydrogen.

DE-A 195 30 993 describes a process for the production of unsaturated cyclic ethers over platinum-doped, cobalt-containing supported catalysts.

DE-A 19,803,368 describes a process for the production of unsaturated cyclic ethers over supported catalysts doped with sulfur and containing cobalt and noble metal, by means of which process satisfactory service lives of the catalyst, but only unsatisfactory space-time yields (STY), are achieved.

In view of the prior art, it is an object of the present invention to provide a process for the production of unsaturated cyclic ethers from diols which gives good space-time yields.

According to the invention, this object is achieved in a novel and improved process for the production of unsaturated cyclic ethers of the general formula I

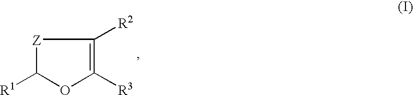

in which

Z denotes —(CHR⁴)_{q}— or —(CHR⁴)_{q}—O—,

q is 0, 1, 2, or 3,

R¹, R², R³, and R⁴ denote hydrogen or C₁ to C₄ alkyl,

by conversion of diols of the general formula II

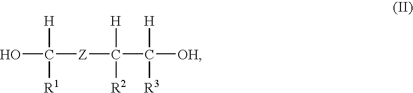

in which Z, R¹, R², and R³, have the aforementioned meanings, in liquid phase at temperatures of from 1500 to 300° C. in the presence of a sulfur-doped, cobalt-containing supported catalyst not activated by reduction before use and containing cobalt and a noble metal applied by sol impregnation to an inert support and selected from the group comprising platinum, palladium, rhodium, iridium, ruthenium, osmium, rhenium, or mixtures thereof, wherein water is added.

The substituents R², R⁴, R⁴, and R⁴, the link Z and the index q in the compounds I and II have the following meanings:

R², R⁴, R⁴, and R⁴ independently denote

hydrogen,

C₁-C₃ alkyl, such as methyl, ethyl, n-propyl, isopropyl, n-butyl, isobutyl, sec-butyl and tert-butyl, preferably C₁-C₃ alkyl, such as methyl, ethyl, n-propyl, and isopropyl, and more preferably methyl and ethyl,

Z stands for (CHR⁴)_{q}— or —(CHR⁴)_{q}—O,

q stands for 0, 1, 2, or 3, preferably 0 or 1, and more preferably 1.

Examples of suitable diols II are 1,4-butanediol, 1,5-pentanediol, 1,6-hexanediol, diethylene glycol, preferably 1,5-pentanediol.

Examples of suitable unsaturated cyclic ethers I are 3,4-dihydro-2H-pyran, 2,3-dihydrofuran, and 1,4-dioxane, preferably 3,4-dihydro-2H-pyran.

In the process of the invention, water is added to supplement the small amount of water of reaction formed during cyclization of the diol (III) to form the cyclic ether (I). The water content is usually adjusted to from 0.01 to 25 wt %, preferably from 0.1 to 20 wt %, and more preferably from 0.5 to 10 wt %, based on diol (II). According to the invention, the water can be added directly to the reaction mixture and/or to the feed stream containing diol (II), also referred to as the “feed”. The addition of water makes it possible to take advantage of the additional benefit of the process of the invention, namely that a diol having a low content of useful material can be used for the process.

The water concentration in the feed or in the reaction mixture can be determined by the Karl Fischer method as specified in DIN 51,777(March 1983).

The process of the invention can be carried out as follows:

To diol (II) there is added from 0.1 to 25 wt % of water, based on diol (II). Alternatively, the water can be directly added to the reaction mixture in the same concentration. This diol (II)-containing feed can usually be caused to react over from 0.2 to 20 wt %, preferably from 0.3 to 10 wt %, of sulfur-doped, cobalt-containing supported catalyst at temperatures ranging from 150° to 300° C., preferably from 160° to 240° C. The cobalt-containing supported catalyst can be used as initial batch or it can be added during the reaction in stepped proportions. The reaction mixture should be uniformly mixed during the reaction, the energy input being set to a value which restricts the space velocity. The water content of the reaction mixture can be monitored during the reaction by determining the water concentration in samples taken from the reaction mixture, and corrected, if need be, by the addition of more water to the reaction mixture. The resulting mixture of the unsaturated cyclic ether (I) and the water (water added plus water of reaction) can be removed by distillation either batchwise or, preferably, continuously. The unsaturated cyclic ether formed during the reaction can optionally be stripped, in order to remove the hydrogen formed during the reaction, using gases which are inert under the conditions of the reaction, such as nitrogen or argon. When the process is carried out continuously, the liquid level in the reaction vessel can be maintained by introducing fresh hydrous diol (II) feed. The addition of alkali metal and/or alkaline earth metal compounds in order to lower the content of saturated cyclic ether that is difficultly separable, by distillation, from the unsaturated 3,4-dihydro-2H-pyran, is not necessary in the process of the invention.

Suitable sulfur-doped supported catalysts containing cobalt and noble metal, are the oxides of cobalt or metallic cobalt and one or more noble metal elements selected from the group comprising platinum, palladium, rhodium, iridium, ruthenium, osmium, rhenium, or mixtures thereof, preferably platinum, palladium, rhenium, or mixtures thereof, and more preferably platinum or palladium, or mixtures thereof, and optionally from 0.001 to 10 wt %, preferably from 0.1 to 5 wt %, and more preferably from 0.5 to 3 wt % of basic alkali or alkaline earth metal salts, scandium, vanadium, chromium, manganese, iron, nickel, copper, zinc, germanium, tin, lead, antimony, bismuth, or mixtures thereof (compound A), preferably lithium, potassium, sodium, calcium, strontium, manganese, iron, nickel, copper, zinc, tin, antimony, or mixtures thereof, and more preferably potassium, sodium, manganese, iron, nickel, copper, zinc, or mixtures thereof, on a porous support.

The percentage by weight of the cobalt in the supported catalyst is usually from 1 to 70 wt %, preferably from 5 to 50 wt % and more preferably from 10 to 40 wt %.

The percentage by weight of the elements selected from the group comprising platinum, palladium, rhodium, iridium, ruthenium, osmium, or rhenium, preferably platinum, palladium, or rhenium, is from 0.001 to 2 wt %, preferably from 0.05 wt % to 1 wt %, and more preferably from 0.01 to 0.5 wt %, based on the supported catalyst.

The percentage by weight of sulfur (calculated as S) is between 0.015 and 2 wt %, preferably from 0.1 to 1 wt %, and more preferably from 0.3 to 0.6 wt %, based on the supported catalyst.

In order to determine the sulfur content of the catalysts, the supported catalyst is dissolved in hydrochloric acid and treated mit hypophosphorous acid. The hydrogen sulfide thus formed is expelled in an N₂ stream, collected in ammoniacal cadmium acetate solution and determined iodometrically.

The supported catalysts usually exhibit a ratio, by weight, of cobalt to the noble metal of from 10:1 to 10,000:1 and of noble metal to sulfur of from 100:1 to 1:100.

Suitable supports are inert supports, such as SiO₂, Al₂O₃, TiO₂, ZrO₂, zeolites of all types, such as zeolites of fine porosity, eg, α-zeolite, zeolites of medium porosity, eg, ZSM 5, ZSM 11, ferrierite, large-pored zeolites, eg, faujasites, β-zeolites, mordenite, offretite, hydrothermally produced phosphates, such as AlPO and SAPO, activated carbons or alkaline earth metal oxides, preferably SiO₂, ZrO₂, and zeolites, and more preferably SiO₂, whilst the ratio, by weight, of cobalt to SiO₂ in the supported catalyst is usually from 1:20 to 1:1.

The supported catalysts usually exhibit a surface area (BET) of from 1 to 600 m²/g, preferably from 10 to 500 m²/g, and more preferably from 50 to 400 m²/g.

The porosity of the supported catalysts is usually from 0.01 to 1.5 mL/g, preferably from 0.1 to 1.2 mL/g, and more preferably from 0.2 to 1 mL/g.

The supported catalysts used in the process of the invention are produced by applying first of all cobalt, then the noble metal in the form of a sol, to the support, followed by sulfur-doping.

Production of the cobalt-containing supported catalysts is well known. Advantageously, impregnation of the porous support material with a soluble cobalt compound (eg, a nitrite, nitrate, sulfite, sulfate, carbonate, hydroxide, of carboxylates, halides, halites, halates, etc.), is optionally carried out simultaneously with or following application of a similarly soluble compound A (eg, a nitrite, nitrate, sulfite, sulfate, carbonate, hydroxide, carboxylates, halides, halites, halates, etc.), followed by thermal decomposition of the anion to form the oxide. Another possibility comprises mixing a cobalt compound with the support material (dry or in suspension, particularly by spray drying), optionally at the same time as a chemical compound A, densifying the material (eg, by kneading, optionally with the addition of a suitable shaping agent), shaping by extrusion, and drying, followed by calcination at temperatures ranging from 200° to 1300° C., preferably from 300° to 1000° C., and more preferably from 400° to 800° C.

The noble metal—selected from the group comprising platinum, palladium, rhodium, iridium, ruthenium, osmium, rhenium, or mixtures thereof—is then applied to the support by spraying the hot support with a preformed sol or by impregnating the support with said sol, before which process any agglomerated catalyst composition produced by previous impregnation is ground down.

The noble metal sol is colloidal material and can be produced by known methods, eg, starting from metal salts in which the noble metal is present at a level of oxidation greater than zero. For example, aqueous solutions of the chlorides, acetates or nitrates of the metal can be used. However, other noble metal salts may be useful and there is no restriction as regards the anion. The reducing agents used can be organic compounds, such as ethanol, methanol, carboxylic acids, and their alkali-metal salts, and also inorganic compounds, such as N₂H₄ or NaBH₄. Preference is given to hydrazine (N₂H₄) and ethanol. The particle size of the metal particles in the sol is governed by the strength of the reducing agent used and by the metal salt used. The sols can be stabilized by the addition of organic polymers, such as polyamines, polyvinylpyrrolidone, or polyacrylates, polyvinyl pyrrolidone (PVP) being preferred, or the preparation of the sol can be carried out by other methods described in the literature. For example, Boennemann et al. (Angew. Chemie, 103 (1991), 1344) describe the manufacture of stable metal sols by reduction of metal salts with (C₈H₁₇)₄N[BEtH₃].

Application of the sols to the support can be carried out by various techniques, which, like the basic synthesis of the catalysts and the catalysts themselves, are described in detail in DE 198 03 368, which is included herein by reference.

It is an advantage that the catalysts disclosed by DE 198 03 368 do not have to be activated prior to use in the process of the invention by treatment with hydrogen or other reducing agents, such as hydrazine.

The unsaturated cyclic ethers I are valuable protective groups for alcohols.

### EXAMPLES

Production of Catalyst A

3400 mL of a solution of 3.25 kg of Co(NO₃)₂.6H₂O in water was stirred with 2.5 kg of SiO₂ powder (water absorption 1.5 mL/g) over a period of ca 2 h, dried for 16 h at 120° C. and calcined for 2 h at 500° C.

This composition was then impregnated with 2.9 L of a noble metal sol, produced by mixing 14.2 g of platinum nitrate in 4.5 L of distilled water with 32 g of polyvinylpyrrolidone and 1.93 L of ethanol and heating under reflux for a period of 4 h, and was then dried at 100° C. in vacuo and calcined for 2 h at 500° C. under a blanket of nitrogen. The catalyst thus produced contained 0.12 wt % of PtO₂.

4.6 g of this composition containing cobalt and platinum were then impregnated with a solution of (NH₄)₂S in water, and then dried at 100° C. in vacuo and calcined for 2 h at 350° C.

Further details on the production and properties of catalyst 1 are given in Table 2.

TABLE 1

| | Amount of | Content of | | Sulfur |
|---|---|---|---|---|
| Cata- | noble metal | noble metal | (NH₄)₂S | content |
| lyst | sol [mL] | [wt %] | [g] | [wt %] |
| A | 2900 | 0.10 | 0.04 | 0.34 |

### EXAMPLES 1 TO 4

1.5 L of 1,5-pentanediol, which, following the addition of water, exhibited a water content as shown in Table 1, and 45 g of catalyst A were used as initial batch. The mixture was heated to the bottom limit of the temperature range with stirring, at which point the reaction started with generation of hydrogen. The resulting 3,4-dihydro-2H-pyran/water mixture was continuously removed by distillation and the bottoms temperature was regulated during the reaction in such a manner that the distillation rate remained constant (from 40 to 50 mL/h). The mixture was simultaneously continuously replenished with hydrous 1,5-pentanediol having a water content as shown in Table 1, within the times revealed in Table 4, in order to keep the level in the reaction flask constant. Following phase separation of the distillate, there was obtained 3,4-dihydro-2H-pyran. The respective space-time yields are listed in Table 1.

### COMPARATIVE EXAMPLE C5

1.5 L of 1,5-pentanediol and 45 g of catalyst A were used as initial batch and heated to the bottom limit of the temperature range with stirring, at which point the reaction started with generation of hydrogen. The reaction scheme differed from Examples from 1 to 4 of the invention in that no water was added. The experiment was carried out in the same manner as that described in DE 19,803,368. Following phase separation of the distillate, there was obtained 3,4-dihydro-2H-pyran. The space-time yield found is given in Table 1.

TABLE 2

| | | | | | Water content | |
|---|---|---|---|---|---|---|
| | | | | Amount of | [wt %], based | |
| | Cata- | Tempera- | Time | added wa- | on 1,5-penta- | STY |
| Ex. | lyst | ture [° C.] | [h] | ter [g] | nediol, | [kg/L · h] |
| 1 | A | 170-230 | 29 | 27.6 | 2 | 0.1 |
| 2 | A | 170-230 | 51 | 54.3 | 3 | 0.12 |
| 3 | A | 170-230 | 52 | 115.5 | 5 | 0.09 |
| 4 | A | 170-230 | 83 | 268.2 | 10 | 0.07 |
| C5 | A | 170-230 | 780 | — | * | 0.021 |

STY = space-time yield
* = no added water, only water of reaction

The test results listed in Table 1 clearly show that the addition of water as proposed in the present invention leads to distinctly better space-time yields, cf Examples 1 to 4.

1. A process for the production of an unsaturated cyclic ether of the general formula I 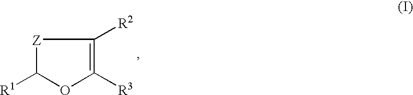
in which
Z denotes —(CHR⁴)_{q}— or —(CHR⁴)_{q}—O—,
q is 0, 1, 2 or 3 and
R¹, R², R³, and R⁴ denote hydrogen or C₁-C₄ alkyl
by conversion of a diol of the general formula II 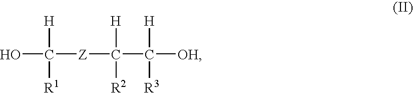
in which Z, R¹, R² and R³ have the aforementioned meanings, in liquid phase at temperatures of from 150° to 300° C. in the presence of a sulfur-doped, cobalt-containing supported catalyst not activated by reduction before use and containing cobalt and a noble metal applied to an inert support by sol impregnation and selected from the group comprising platinum, palladium, rhodium, iridium, ruthenium, osmium, rhenium, or mixtures thereof, wherein water is added.

2. A process for the production of an unsaturated cyclic ether as defined in claim 1, wherein water is added to the feed and/or to the reaction mixture during the reaction.

3. A process for the production of an unsaturated cyclic ether as defined in claim 1 or claim 2, wherein the water is added so as to maintain a water content of from 0.01 to 25 wt %, based on diol (II).

4. A process for the production of an unsaturated cyclic ether I as defined in any of claims 1 to 3, wherein the water content is from 0.1 to 20 wt %, based on diol (II).

5. A process for the production of an unsaturated cyclic ether I as defined in any of claims 1 to 4, wherein the water content is from 0.5 to 10 wt %, based on diol (II).

6. A process for the production of an unsaturated cyclic ether I as defined in any of claims 1 to 5, wherein the cobalt-containing supported catalysts contain from 1 to 70 wt % of cobalt, from 0.001 to 2 wt % of one or more noble metals and from 0.015 to 2 wt % of sulfur.

7. A process for the production of an unsaturated cyclic ether as defined in any of claims 1 to 6, wherein 1,5-pentanediol is converted to 3,4-dihydro-2H-pyran.
